(19) United States
(12) Patent Application Publication
(10) Pub. No.: US 2007/0196288 A1
(43) Pub. Date: Aug. 23, 2007

(54) Method of treating periodontitis and of reducing dentinal sensitivity

(75) Inventors: Sasi Kumar Sunkara, Buffalo, NY (US); Sebastian G. Ciancio, Eggertsville, NY (US); Federick Sachs, Buffalo, NY (US); Hakimuddin T. Sojar, Amherst, NY (US)

Correspondence Address: PHILLIPS LYTLE LLP;INTELLECTUAL PROPERTY GROUP, 3400 HSBC CENTER, BUFFALO, NY, 14203-3509, US

(73) Assignee: Research Foundation of the State University of New York

(21) Appl. No.: 11/708,731

(22) Filed: Feb. 21, 2007

Related U.S. Application Data
(60) Provisional application No. 60/776,117, filed on Feb. 23, 2006.
(60) Provisional application No. 60/817,546, filed on Jun. 29, 2006.

(51) Int. Cl. A61K 8/19; A61K 33/24; A61K 47/02

(52) U.S. Cl. 424/53; 424/49; 424/617; 514/789

(57) ABSTRACT

An improved method of treating periodontitis includes the steps of: creating a formulation containing a lanthanide-containing protease inhibitor; and applying the formulation to a periodontal area. The lanthanide may be cerium chloride, samarium chloride, lutetium chloride, cerium chloride heptahydrate, lutetium chloride hexahydrate, samarium chloride hexahydrate, gadolinium, gadolinium nitrate, gadolinium chloride, gadolinium chloride hexahydrate and gadolinium nitrate hexahydrate. The invention also provides an improved method of reducing dentinal sensitivity, including the steps of: creating a formulation containing at least one of gadolinium and a gadolinium-containing compound; and applying the formulation to a sensitive dentinal area. The formulation may be a paste, a solution or a powder.

### CROSS-REFERENCE TO RELATED APPLICATIONS

This application claims the benefit of pending provisional application Ser. No. 60/776,117, filed Feb. 23, 2006, and entitled, “Materials and Method to Reduce Dentinal Hypersensitivity”, and Ser. No. 60/817,546, filed Jun. 29, 2006, and entitled, “Inhibition of Proteases”, which applications are incorporated herein by reference in their entireties.

### TECHNICAL FIELD

The present invention relates generally to periodontitis and dentinal sensitivity, and, more particularly, to improved methods of treating periodontitis and of reducing dentinal sensitivity.

### BACKGROUND ART

Host and bacterial proteases play a vital role in periodontitis. Inhibitors of these proteases are necessary for control of this disease.

Understanding of the molecular basis of disease progression and the role of various virulence factors in the multi-factorial disease, periodontitis, has led to the development of various treatment modalities for specific elements of the disease. Oral bacterial pathogens, particularly, Porphyromonas Gingivalis (“P. Gingivalis”), are one of the major etiological agents involved in gingivitis and advanced adult periodontitis. [White, D. & Mayrand, D., “Association of Oral Bacteroides with Gingivitis and Adult Periodontitis”, 16 Journal of Periodontal Research, 259-265 (1981); Moore, W. E., Holdeman, L. V., Smibert, R. M. et al., “Bacteriology of Severe Periodontitis in Young Adult Humans, 38 Infect. Immun. 1137-1148 (1982).]

The virulence factors, such as lipopolysaccharide, fimbriae, haemagglutinins, vesicles and proteases, of periodontis have been studied and characterized. [Slots, J. & Genco, R. J., “Black-Pigmented Bacteroides Species, Capnocytophaga Species, and Actinobacillus Actinomycetemcomitans in Human Periodontal Disease: Virulence Factors in Colonization, Survival, and Tissue Destruction”, 63 J. Dent. Res. 412-421 (1984).]

Of the various virulence factors, great emphasis has been placed on bacterial proteases. [Smalley, J. W., Birss, A. J., Kay, H. M. et al., “The Distribution of Trypsin-Like Enzyme Activity in Cultures of a Virulent and an Avirulent Strain of Bacteroides Gingivalis W50”, 4 Oral Microbiology & Immunology, 178-181 (1989); Marsh, P. D., McKee, A. S., McDermid, A. S. et al., “Ultrastructure and Enzyme Activities of a Virulent and an Avirulent Ariant of Bacteroides Gingivalis W50”, 50 FEMS Microbiol. Lett. 181-185 (1989); Grenier, D. & Mayrand, D., “Selected Characteristics of Pathogenic and Nonpathogenic Strains of Bacteroides Gingivalis”, 25 Journal of Clinical Microbiology 738-740 (1987).]

A number of physiologically-significant proteins are hydrolyzed by proteases from this microorganism. [Kuramitsu, H. K., “Proteases of Porphyromonas Gingivalis: What Don't They Do?”, 13 Oral Microbiol. Immunol. 263-270 (1998); Chen, Z., Potempa, J., Polanowski, A. et al., “Purification and Characterization of a 50-kDa Cysteine Proteinase (Gingipain) from Porphyromonas Gingivalis”, 267 J. Biol. Chem. 18896-18901 (1992).]

New generation agents are being used for treatment. These target expression and the effects of specific virulence factors. Oral microbial ecology consists of various bacterial species, involving pathogenic and normal microbial flora. [Gibbons, R. J. & Houte, J. V., “Bacterial Adherence in Oral Microbial Ecology”, 29 Annu. Rev. Microbiol., 19-44 (1975).] Using an agent that affects prokaryotes in general might lead to disturbances in the ecological balance and might increase the risk of opportunistic infections. Inhibition of proteases may have therapeutic value not only because it limits their direct and indirect damage to the host, but also it interferes with a critical component of the pathogen's survival. Loss of protease function may render the organism more susceptible to control by the endogenous defense mechanisms of the host. Hence, proteases are excellent targets for the rational design of drugs with a potential to cure and prevent periodontitis. Many substances have been identified as inhibitors of proteases from P. Gingivalis. These include N-ethylmaleimide, iodoacetic acid, and iodoacetamide. [Bedi, G. S. & Williams, T., “Purification and Characterization of a Collagen-Degrading Protease from Porphyromonas Gingivalis”, 269 J. Biol. Chem. 599-606 (1994).]

Calcium is known for its role on proteases. It is know to have a significant effect on the stability of Gingipain. [Mellgren, R. L., “Calcium-Dependent Proteases: An Enzyme System Active at Cellular Membranes?”, 1 Faseb. J. 110-115 (1987); [Eichinger, A., Beisel, H. G., Jacob, U. et al., “Crystal Structure of Gingipain R: An Arg-Specific Bacterial Cysteine Proteinase with a Caspase-Like Fold”, 18 Embo. J 5453-5462 (1999); Nakayama, K., “Domain-Specific Rearrangement Between the Two Arg-Gingipain-Encoding Genes in Porphyromonas Gingivalis: Possible Involvement of Nonreciprocal Recombination”, 41 Microbiol. Immunol. 185-196 (1997).]

Lanthanide ion sizes, bonding, coordinating geometry, and donor atom preference, are similar to calcium. [Turro, C., Fu, P. K. & Bradley, P. M., “Lanthanide Ions as Lluminescent Probes of Proteins and Nucleic Acids”, 40 Met. Ions. Biol. Syst. 323-353 (2003).]

Certain enzymes which require Ca²⁺ are inhibited or activated by lanthanide (Ln³⁺) ions through the formation of an abortive enzyme-substrate complex. [Evans, C. H., “Interactions of Tervalent Lanthanide Ions with Bacterial Collagenase (Clostridiopeptidase A)”, 195 Biochem. J. 677-684 (1981).]

The possible use of lanthanides as functional replacements for Ca²⁺ has been demonstrated in enzymatic reactions. They activate the conversion of trypsinogen into trypsin, but inhibit staphylococcal nuclease. [Darnall, D. W. & Birnbaum, E. R., “Rare Earth Metal Ions as Probes of Calcium Ion Binding Sites in Proteins. Neodymium (3) Acceleration of the Activation of Trypsinogen”, 245 J. Biol. Chem. 6484-6486 (1970); Furie, B., Eastlake, A., Schechter, A. N. et al., “The Interaction of the Lanthanide Ions with Staphylococcal Nuclease”, 248 J. Biol. Chem. 2821-2825 (1973).]

As with bacterial α-amylase, there may be activation at low lanthanide ion concentrations, but inhibition at high concentrations due to non-specific binding. [Darnall, D. W. & Birnbaum, E. R., “Lanthanide Ions Activate Alpha-Amylase”, 12 Biochemistry, 3489-3491 (1973).] Use of lanthanides might help to specifically target virulence factors like bacterial proteases, rather than the whole microbial flora

Dentinal sensitivity may occur after placing fillings or on exposed dentin. Other products marketed for reducing dentinal sensitivity use glutaraldehyde (a poison) or solutions with high acidic pH. Gadolinium nitrate in combination with water is less acidic (i.e., has a pH of about 4.3 as compared to other marketed products having a pH of about 2).

Materials that reduce dentinal hypersensitivity produce their effect either by blocking the dentinal tubules or by soothing the nerve. Gadolinium (Gd) is also known for its effect on mechanogated ion channels.

Therefore, there is a need for improved methods for treating periodontitis and for reducing dentinal sensitivity.

### DISCLOSURE OF THE INVENTION

With reference to the corresponding parts, portions or surfaces of the disclosed embodiment, merely for purposes of illustration and not by way of limitation, the present invention broadly provides improved methods for treating periodontitis and of reducing dentinal sensitivity.

In one aspect, the invention provides an improved method of treating periodontitis, comprising the steps of: creating a formulation containing a protease inhibitor; and applying this formulation to a periodontal area.

The formulation may be a solution, a paste or a powder.

The protease inhibitor may contain at least one lanthanide, such as (but not limited to) a lanthanide selected from the group consisting of cerium chloride, samarium chloride, lutetium chloride, cerium chloride heptahydrate, lutetium chloride hexahydrate, samarium chloride hexahydrate, gadolinium and a gadolinium-containing compound.

The gadolinium-containing compound may be selected from the group consisting of gadolinium nitrate, gadolinium chloride, gadolinium chloride hexahydrate and gadolinium nitrate hexahydrate.

In another aspect, the invention provides an improved method of reducing dentinal sensitivity, comprising the steps of: creating a formulation containing at least one of gadolinium and a gadolinium-containing compound; and applying this formulation to a sensitive dentinal area. The concentration of gadolinium or a gadolinium-containing compound may be from about 1.5% to about 6%.

The formulation may be a solution, a paste or a powder.

The formulation may be a solution containing gadolinium nitrate, and may have a pH of about 4.3.

The solution may contain at least one of water, sulfasalicyclic acid, sodium lauryl sulfate and alcohol. More particularly, the solution may contain sulfasalicyclic acid and water, or sulfasalicyclic acid and alcohol.

Thus, the general object of the invention is to provide an improved method of treating periodontitis.

Another object is to provide an improved method of reducing dentinal sensitivity.

These and other objects and advantages will become apparent from the foregoing and ongoing written specification, the drawings and the appended claims.

### BRIEF DESCRIPTION OF THE DRAWINGS

FIG. 1 is a plot of absorbance (ordinate) vs. concentration of protease inhibitor (abscissa), showing the effect of various lanthanides on cell culture media-precipitated proteins.

FIG. 2 is a plot of absorbance (ordinate) vs. concentration of protease inhibitor (abscissa), showing the effect of various lanthanides on cell surface-extracted proteins.

FIG. 3 are two stains showing the effect of gadolinium on cell culture media proteins, with gelatin zymography.

FIG. 4 are two stains showing the effect of gadolinium on cell surface-extracted proteins, with gelatin zymography.

FIG. 5 is an SEM image of a dentine surface after application of gadolinium nitrate dissolved in water.

FIG. 6 is an SEM of the dentinal tubule of the dentinal slice shown in FIG. 5.

FIG. 7 is a backscattered image of the SEM shown in FIG. 6.

FIG. 8 is an SEM image of the dentinal tubule after application of SuperSeal (potassium oxalate).

FIG. 9 is an SEM image of the dentinal tubule after application of gadolinium dissolved in water and sulfasalicyclic acid.

FIG. 10 is a back scattered image of the dentinal tubule shown in FIG. 9, with the gadolinium compound appearing as bright areas.

FIG. 11 is an SEM image of the dentine surface after application of gadolinium dissolved in sulfasalicyclic acid and alcohol.

FIG. 12 is an SEM of the dentinal tubule of the dentinal slice shown in FIG. 11.

FIG. 13 is a back scattered image of the dentinal tubule shown in FIG. 12, with the gadolinium compound appearing as bright areas.

### DESCRIPTION OF THE PREFERRED EMBODIMENTS

At the outset, it should be clearly understood that like reference numerals are intended to identify the same structural elements, portions or surfaces consistently throughout the several drawing figures, as such elements, portions or surfaces may be further described or explained by the entire written specification, of which this detailed description is an integral part. Unless otherwise indicated, the drawings are intended to be read together with the specification, and are to be considered a portion of the entire written description of this invention. As used in the following description, the terms “horizontal”, “vertical”, “left”, “right”, “up” and “down”, as well as adjectival and adverbial derivatives thereof (e.g., “horizontally”, “rightwardly”, “upwardly”, etc.), simply refer to the orientation of the illustrated structure as the particular drawing figure faces the reader. Similarly, the terms “inwardly” and “outwardly” generally refer to the orientation of a surface relative to its axis of elongation, or axis of rotation, as appropriate.

### Material and Methods re Treatment of Periodontitis

### Bacterial Strains and Growth Conditions

P. Gingivalis 381 was grown in a brain heart infusion broth (EMD Chemicals Inc.) supplemented with 5 μg of hemin, and vitamin K, pH 7.4, at 37° C. for 2 days in a Form a anaerobic chamber (85% N₂, 10% H₂, 5% CO₂).

### Isolation of Proteins

All steps in the isolation of P. Gingivalis proteins were carried out at 4° C. Proteins isolated were obtained from 2-day-old culture of P. Gingivalis, strain 381. The cells and culture media were separated by centrifugation at 10,000×g for 10 minutes in a Sorvall centrifuge.

### Extraction of Cell Surface Proteins

P. Gingivalis cells collected after centrifugation were washed three times with 50 millimolar (“mM”) HEPES buffer, pH 7.0. The washed cells were stirred with 1% Triton X-100 (Fisher Scientific) dissolved in HEPES (Fisher Scientific) at 4° C. for 15 minutes to solubilize cell surface proteins. The cells were centrifuged at 10,000×g in a Sorvall centrifuge for 10 minutes, and the supernatant was collected. [Sojar, H. T., Lee, J. Y., Bedi, G. S. et al., “Purification and Characterization of a Protease from Porphyromonas Gingivalis Capable of Degrading Salt-Solubilized Collagen”, 61 Infect. Immun. 2369-2376 (1993).] The amount of protein was estimated using a Spectrophotometer ND-1000 by absorbance at 280 nm.

### Precipitation of Proteins from Bacterial Culture Media

To the cell-separated culture supernatant, ammonium sulfate was added to achieve 80% saturation in a volume of 400 ml. After 4 hours, the precipitate was collected by centrifugation at 11,000×g for 45 minutes. It was then dissolved in HEPES and dialyzed against 4 liters of HEPES buffer. The precipitate formed on dialysis was removed by centrifugation at 10,000×g for 30 minutes, the supernatant was collected, and the precipitated material was discarded. Extracted proteins was analyzed for protein content using a Spectrophotometer ND-1000.

### Inhibitory Substances

Different lanthanide group elements (Sigma-Aldrich Corporation) were selected to assess their ability to inhibit proteases from P. Gingivalis. These include cerium chloride heptahydrate (CeCl₃.7H₂O), 99.9%; lutetium chloride hexahydrate (LuCl₃.6H₂O), 99.9%; samarium chloride hexahydrate, (SmCl₃.6H₂O), ≧99%; gadolinium chloride hexahydrate (GdCl₃.6H₂O), 99%; and gadolinium nitrate hexahydrate, (Gd(NO₃)₃.6H₂O), 99.9%.

### Enzyme Assays

Activity of cell surface-extracted and culture-precipitated proteases was assayed by using the synthetic chromogenic substrate benzoyl-L-Arg-p-nitroanilide (BAPNA). [Erlanger, B. F., Kokowsky, N. & Cohen, W., “The Preparation and Properties of Two New Chromogenic Substrates of Trypsin”, 95 Arch. Biochem. Biophys., 271-278 (1961).] Colorimetric values measured as absorbance were obtained using Spectronic 2000 Methodology for culture media-precipitated proteins.

The reaction mixture consisted of 50 μl of 100 mM substrate in 50 mM HEPES buffer, pH 7, and 25 μl of the sample protease containing 2 mg/ml of protein was used to test chromogenic activity of the substrate. With 5% of 2-mercaptoethanol as reducing agent, volume was made to 250 μl. Various concentrations of the inhibitor 2 mM; 4 mM; 6 mM; 8 mM; 10 mM were used.

### Methodology for Cell Surface-Extracted Proteins

The reaction mixture consisted of 100 μl of 100 mM substrate in 50 mM HEPES buffer, pH 7, 50 μl of an approximately 1 mg/ml of proteins was used to test chromogenic activity of the substrate. With 5% of 2-mercaptoethanol as reducing agent, the volume was made to 500 μl. Various volumes of the inhibitor 0.4 mM; 0.8 mM; 1.2 mM; 1.6 mM; 2.0 mM; 2.4 mM were used.

After incubation at 37° C. for 1 hour, 100 μl of 5 N acetic acid was added to stop the reaction in both experimental samples. The total volume was made to 1 ml by addition of water. The p-nitroaniline released was determined by measuring A405.

### Zymography

Since P. Gingivalis proteases cleave gelatin effectively, the gelatin-cleaving activity of the extracted proteins was checked by zymography, as described by Heussen and Dowdle using 10% Zymogen (Gelatin) Gel 1.0 mm×10 well (Invitrogen Corporation). [Heussen, C. & Dowdle, E. B., “Electrophoretic Analysis of Plasminogen Activators in Polyacrylamide Gels Containing Sodium Dodecyl Sulfate and Copolymerized Substrates”, 102 Anal. Biochem. 196-202 (1980).]

The extracted proteins, with the reducing sample buffer containing 5% mercaptoethanol, was electrophoresed. After electrophoresis, the gel was washed in 50 mM HEPES, pH 7.0, containing 2% Triton X-100 for 30 minutes, and rinsed twice with the same buffer without Triton X-100. The gel was then cut into 3 parts before transferring to a development buffer containing 50 mM HEPES, 5% 2-mercaptoethanol, buffered at pH 7.0. One part was incubated with developing buffer whereas the other two parts were incubated in the presence of inhibitor with varying concentrations of 5 mM and 10 mM. The gel was incubated at 37° C. overnight on a rocker. The incubated gel was then fixed with 50% methanol and 12% acetic acid for 30 minutes, and stained with 0.1% Coomassie blue in 50% methanol and 12% acetic acid to visualize lytic bands.

### Results

### Hydrolysis of Chromogenic Substance BAPNA

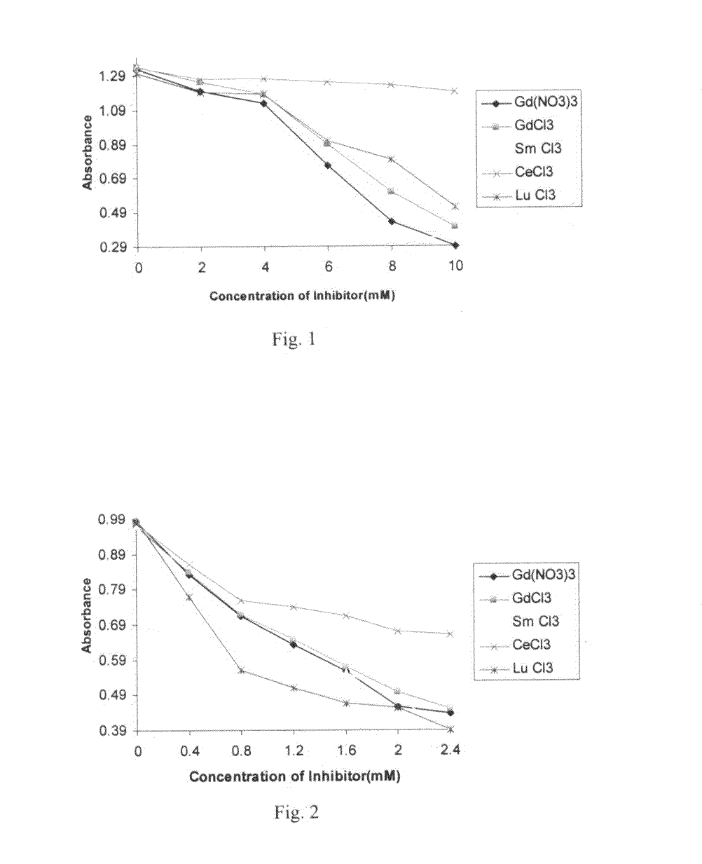

BAPNA was hydrolyzed in the presence of cell surface-extracted and culture media-precipitated proteins. The presence of 2-mercaptoethanol was necessary for the activity. Concentration-dependent inhibition of BAPNA hydrolysis was observed in the presence of lanthanides. When cell culture-precipitated proteins were incubated with various lanthanide compounds (gadolinium nitrate, gadolinium chloride, samarium chloride, cerium chloride, and lutetium chloride) as inhibitors, protease activity was inhibited by approximately 78, 70, 75, 11 and 60%, respectively, as shown in FIG. 1. The inhibitory effects on cell surface-extracts was on the order of 56, 54, 59, 32, and 60%, respectively, as shown in FIG. 2.

Concentrations varying from of 0.4 mM to 2.4 mM were observed at A405. At 0.4 mM, all inhibitors had approximate OD of 0.8. As the concentrations were increased to 2.4 mM, values approached 0.43 for gadolinium nitrate, 0.45 for gadolinium chloride, 0.39 for samarium chloride, 0.66 for cerium chloride, and 0.39 for lutetium chloride.

Variation due to a change in the anion (gadolinium chloride hexahydrate vs. gadolinium nitrate hexahydrate) was not of great significance. Having been used for many therapeutic purposes, gadolinium has been well studied with respect to its safety in humans. [Bruce, D. W., Hietbrink, B. E. & Dubois, K. P., “The Acute Mammalian Toxicity of Rare Earth Nitrates and Oxides, 5 Toxicol. Appl. Pharmacol., 750-759 (1963).]

The gadolinium compound, gadolinium nitrate, appeared to have significant effect on proteins.

### Gelatin Zymography

Gelatin zymography was used in this study to evaluate the inhibitory effect of gadolinium on collagenolytic activities. Proteins extracted from cell surface and those precipitated from cell culture media were subjected to zymography. High proteolytic activities were detected on the gelatin zymogram in the absence of inhibitor. However, negative staining bands were absent in the presence of an inhibitor gadolinium at a concentration of 5 mM.

### Discussion

Bacterial pathogens produce a wide range of cell surface and secretory proteases. [Toda, K., Otsuka, M., Ishikawa, Y. et al., “Thiol-Dependent Collagenolytic Activity in Culture Media of Bacteroides Gingivalis”, 19 J. Periodontal Res. 372-381 (1984).]

Periodontal disease can be attributed to their action on host connective tissue along with potentiation of inflammatory process that adds to this pathogenicity. Among the potential virulence factors of P. Gingivalis, proteases are the most widely studied. [Potempa, J., Banbula, A. & Travis, J., “Role of Bacterial Proteinases in Matrix Destruction and Modulation of Host Responses”, 24 Periodontol 2000 153-192 (2000).]

In our experiments, even in the presence of an activator (5% mercaptoethanol), we observed inhibition of thiol-dependent proteases. [Toda, K., Otsuka, M., Ishikawa, Y. et al., “Thiol-Dependent Collagenolytic Activity in Culture Media of Bacteroides Gingivalis”, 19 J. Periodontal Res. 372-381 (1984); Bhogal, P. S., Slakeski, N. & Reynolds, E. C., “A Cell-Associated Protein Complex of Porphyromonas Gingivalis W50 Composed of Arg- and Lys-Specific Cysteine Proteinases and Adhesins”, 143 Microbiology (Pt 7) 2485-2495 (1997).]

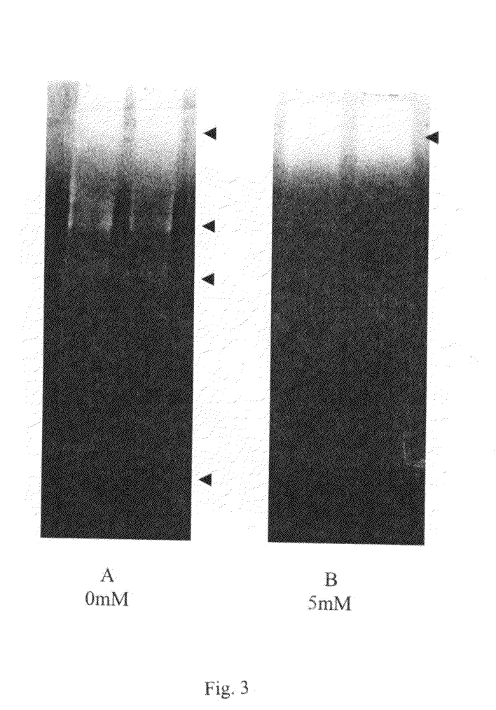

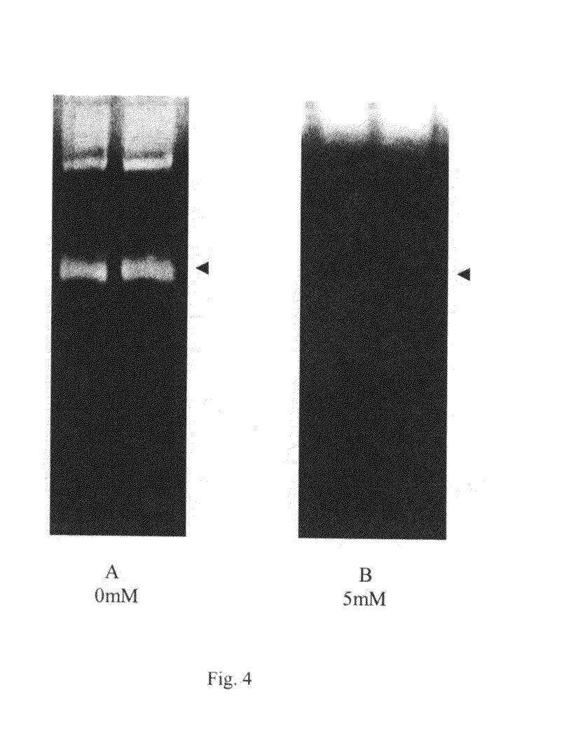

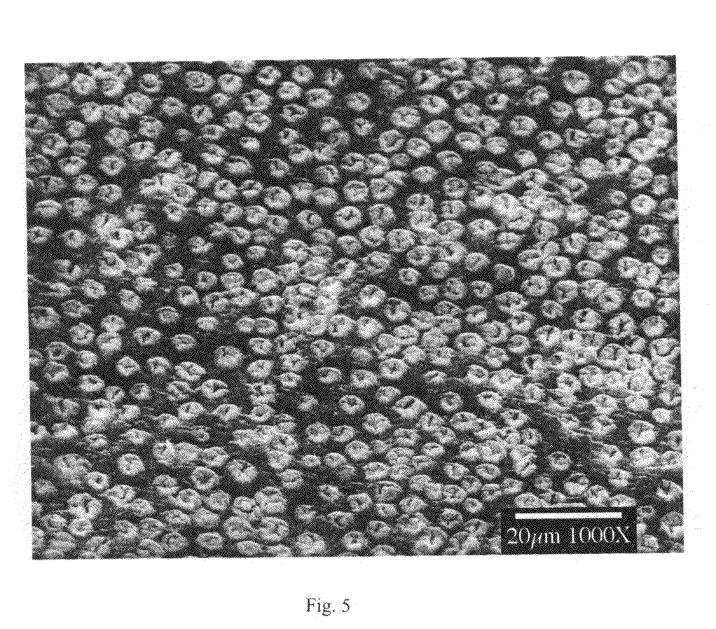

With gadolinium nitrate, there was a 78% inhibition of the cell culture media-precipitated protease at 10 mM, and a 56% inhibition of cell surface extracted protease at 2.4 mM concentrations, respectively (FIGS. 1 and 2). It also inhibited the collagenolytic activity of both the experimental proteins (FIGS. 3 and 4). In FIG. 3A, the zones of enzymatic activity are indicated by negative staining. FIG. 3B shows the absence of zones of enzymatic activity in the presence of an inhibitor at a concentration of 5 mM.

Gingival crevicular fluid at discrete periodontitis sites has revealed the presence of high levels of proteolytic activity. [Potempa, J., Banbula, A. & Travis, J., “Role of Bacterial Proteinases in Matrix Destruction and Modulation of Host Responses”, 24 Periodontol 2000 153-192 (2000).] Detailed studies of this fluid indicated a mixture of host enzymes. Activity of collagenases and chymotrypsin-like proteases is well known. Trypsin-like proteases deteriorate host defenses against pathogens. [Grenier, D., “Degradation of Host Protease Inhibitors and Activation of Plasminogen By Proteolytic Enzymes from Porphyromonas Gingivalis and Treponema Denticola”, 142 Microbiology (Pt 4) 955-961 (1996).]

Selective inhibition of major virulence protease of an organism may affect the survival of that organism, but host and bacterial factors involved in a disease need to be addressed to treat a disease.

Lanthanides are a group of elements about the size of the calcium ion, but with a very high charge. The major ligand for lanthanide ions on proteins is the carboxyl and hydroxyl oxygen. [Wang, R., Liu, H., Carducci, M. D. et al., “Lanthanide Coordination with Alpha-Amino Acids Under Near Physiological pH Conditions: Polymetallic Complexes Containing the Cubane-Like [Ln4(mu3-OH)4]8+Cluster Core”, 40 Inorg. Chem. 2743-2750 (2001).]

Studies have shown the affect of lanthanides on inflammation and enzymes, such as collagenase. [Jancso, N., “Inflammation and the Inflammatory Mechanisms”, 13 J. Pharm. Pharmacol. 577-594 (1961); Evans, C. H. & Ridella, J. D., “Inhibition, by Lanthanides, Of Neutral Proteinases Secreted By Human, Rheumatoid Synovium”, 151 Eur. J. Biochem. 29-32 (1985)].

In our experiments, we observed that gadolinium inhibited most of the thiol protease. This might lead to reduction in the total amount of trypsin-like protease activity. This type of inhibition might not only prevent the direct action of the trypsin-like proteases, but might also enhance the efficacy of host protease inhibitors.

Use of lanthanides is a way to control host- and bacterial-generated proteases. Our results suggested that we should look into the ability of these elements on various proteases in their purified forms, and, in particular to an organism and in a biofilm. Work with this group of elements is most necessary for evidence that lanthanides have effects only on pathogenic organisms with no significant effects on oral microbial ecology. Proteases play a vital role in the survival of pathogenic organisms. There is very high chance that inhibition of proteases may affect the survival of many pathogenic organisms.

Periodontitis is a chronic disease, and host factors along with subgingival plaque play a vital role in the progress of the disease. In order to confirm the lanthanides' potential, an in vivo examination is essential.

### Materials and Methods re Reduction of Dentinal Sensitivity

Caries-free surgically-extracted human molars were cleaned of organic material, and, after removal of the crown, were sectioned mesio-distally to provide one to two 1-mm dentine discs which were used in the experiment immediately. The disks were etched with 37% phosphoric acid for 10-15 seconds and rinsed under tap water. Gadolinium nitrate was placed on the dentine disks for 5-10 minutes before rinsing under flowing tap water. Dentinal discs were dried completely and then split using pliers. Specimens were then carbon coated for examination under a scanning electron microscope. The same procedure was repeated using (1) gadolinium nitrate and alcohol, (2) gadolinium, sulfasalicyclic acid, and alcohol, (3) gadolinium, sulfasalicyclic acid, and water, and (4) gadolinium nitrate, sodium lauryl sulfate and water.

All these samples were tested with gadolinium nitrate/gadolinium in a concentration range from 1.5% to 6%. However, gadolinium and gadolinium nitrate are effective even at concentrations below 1%.

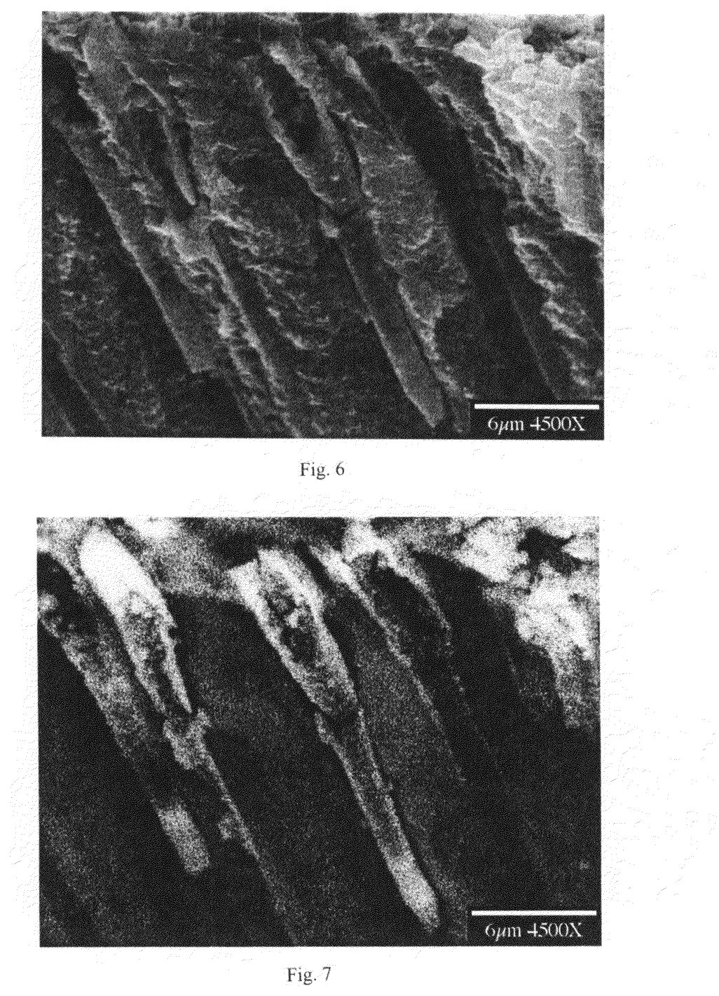

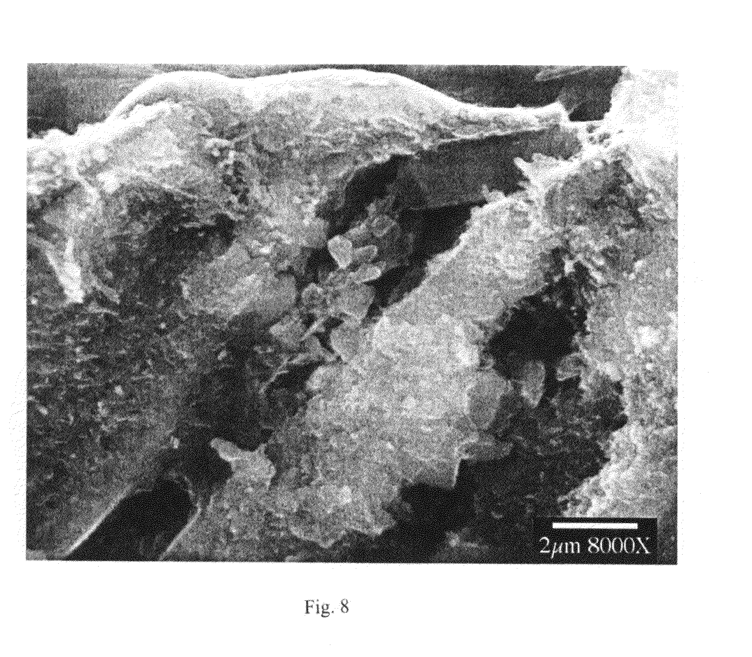

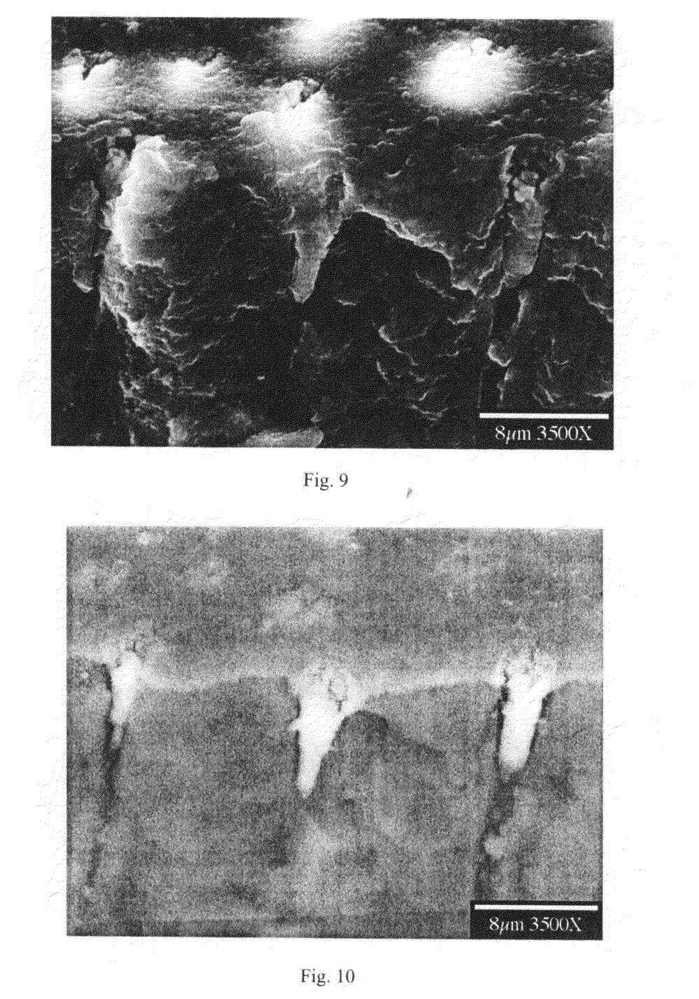

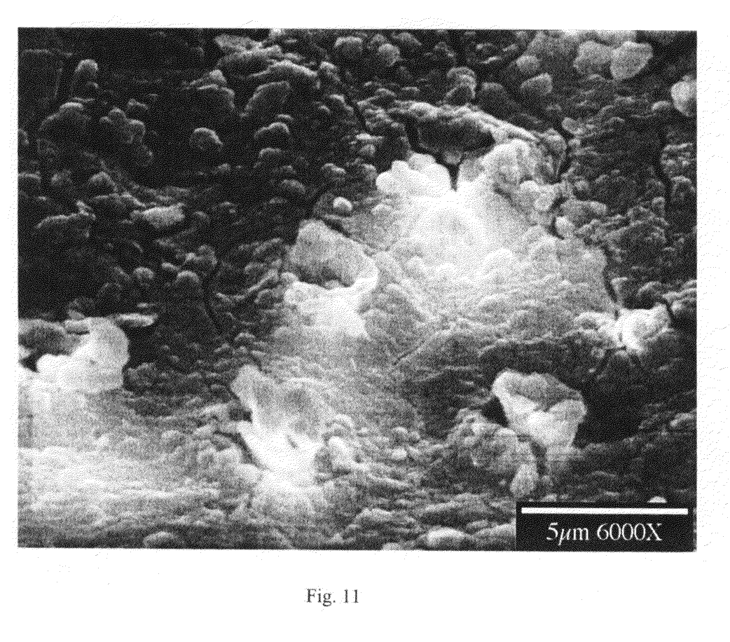

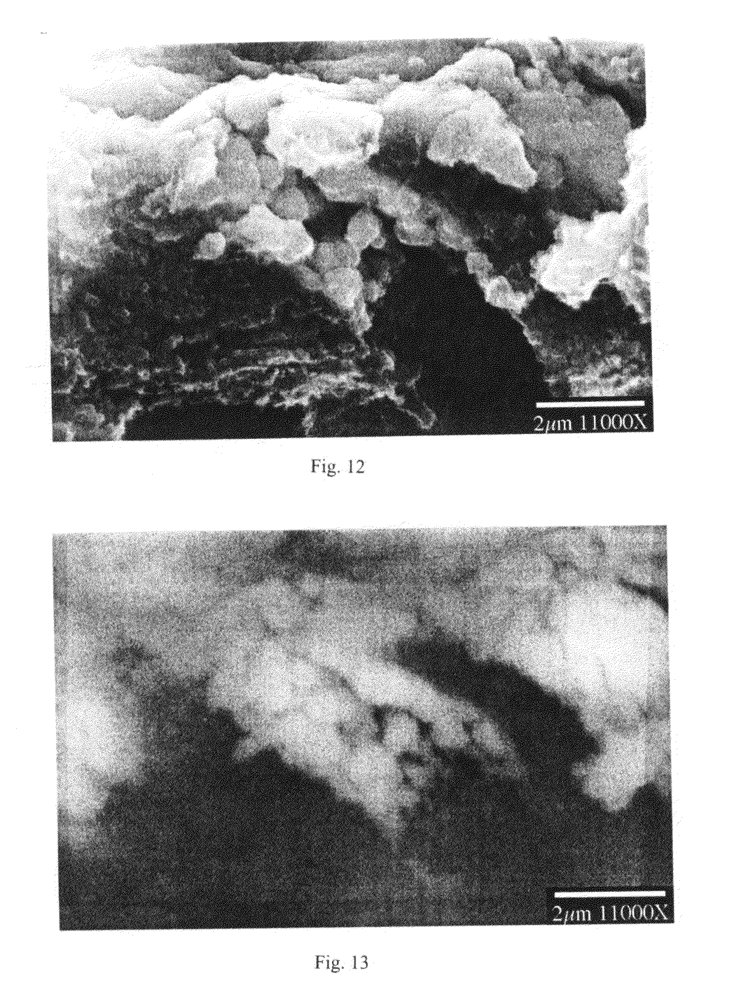

A variation in the procedure was to use slight application of air pressure, during the application of chemicals on the dentine discs and later for drying the surface. We concluded on what material was deposited only after examination with Scanning Electron Microscope and Energy Dispersive X-ray microanalysis (SEM/EDX) of the dentine discs, as shown in FIGS. 6 and 7. SEM/EDX confirmed the presence of gadolinium precipitates on the dentin surface and introduction and occlusion of gadolinium into the dentinal tubules.

Even at pH of 4.3, in vitro studies showed deposition of gadolinium on the surface of the dentine and with in the tubule with gadolinium nitrate. We observed similar pattern of deposition with our control, SuperSeal (potassium oxalate), with a pH of 2.0. Gadolinium is a heavy metal and therefore appears in bright areas on a back-scattered image, as shown in FIGS. 10 and 13.

The plug-like structures formed in close proximity to the tubule walls (FIGS. 6 and 7). Gadolinium in a solution of water and sulfasalicyclic acid appears to alter the hydrodynamics of the tubular fluid more efficiently to reduce dentinal hypersensitivity, than with gadolinium nitrate. The main drawback is its low acidic pH 2. Alcohol with the same combination increased the depth of penetration and the amount of approximation with tubule surface. This invention will work well both as a regular toothpaste and as a cavity liner.

The pH of the material is less acidic, as compared to many materials used to alleviate hypersensitivity. It also leaves significant deposits on the surface after thorough washing under tap water. We hope to see the same effects in our future clinical trials. We are using 6% solutions of gadolinium-based compounds, based on the experimental trials with potassium oxalate. The principal comparison of this technology is with a marketed product containing potassium oxalate. The reaction chemistry of potassium oxalate with calcium phosphate is well known, producing rapid precipitates of calcium oxalate, which superficially occlude dentinal tubules. We have achieved promising results of dentinal occlusion with gadolinium nitrate, and tubule penetration depths greater than potassium oxalate when alcohol is used as a solvent.

Gadolinium oxalate and gadolinium metal both required very acidic conditions for dissolution. We felt that the pH was too low in these cases for clinical application.

With gadolinium nitrate (pH 4.3), we have observed deposits of gadolinium on dentinal surface and within tubule. The pattern of deposition is similar to that of potassium oxalate (pH-2.0). These gadolinium compounds do not have any known adverse health effects. Even though tag-like structures and surface deposits have been observed with gadolinium in a mixture of sulfasalicyclic acid and alcohol or water, the pH of this combination is acidic, similar to SuperSeal, and they do not serve the rationale for using a product for dentinal hypersensitivity.

The material may be used in the following products: dentinal hypersensitivity toothpaste, professional application medicament for treatment of hypersensitivity, cavity liner, varnish, dentin bonding agent, and antimicrobial agents.

Gadolinium is superior to known products because it is less acidic compared to the potassium oxalate. The adverse effects are anticipated to be less than potassium oxalate. Gadolinium has been studied for more than a decade by using it in chelated form as an MRI contrast agent. Gadolinium forms a thin coating extending along the length of the tubule and forms crystals in situ that can close the tubules to inappropriate influx of toxic agents. We anticipate that gadolinium applied with alcohol or other agents modifying surface energy will work much better than potassium oxalate. Gadolinium coats the dentin tubules in a more effective manner than oxalate or glutaraldehyde. Compared with glutaraldehyde, which is a tissue fixative (poison), gadolinium appears to be safe.

While there has been described what is believed to be the preferred embodiment of the present invention, persons skilled in the art will recognize that other and further changes and modifications may be made thereto without departing from the spirit of the invention. Therefore, the invention is not limited to the specific details and representative embodiments shown and described herein. The terminology and phraseology used herein is for purposes of description and should not be regarded as limiting. Accordingly, persons skilled in this art will readily appreciate that various additional changes and modifications may be made without departing from the spirit of the invention, as defined and differentiated in the following claims.

What is claimed is:

1. The method of treating periodontitis, comprising the steps of:
creating a formulation containing a protease inhibitor; and
applying said formulation to a periodontal area.

2. The method as set forth in claim 1 wherein said formulation is one of a solution, a paste and a powder.

3. The method as set forth in claim 1 wherein said protease inhibitor contains at least one lanthanide.

4. The method as set forth in claim 3 wherein said lanthanide is selected from the group consisting of cerium chloride, samarium chloride, lutetium chloride, cerium chloride heptahydrate, lutetium chloride hexahydrate, samarium chloride hexahydrate, gadolinium and a gadolinium-containing compound.

5. The method as set forth in claim 4 wherein said compound is selected from the group consisting of gadolinium nitrate, gadolinium chloride, gadolinium chloride hexahydrate and gadolinium nitrate hexahydrate.

6. The method as set forth in claim 1 wherein said protease inhibitor is selected from the group consisting of cerium chloride, samarium chloride, lutetium chloride, cerium chloride heptahydrate, lutetium chloride hexahydrate, samarium chloride hexahydrate, gadolinium nitrate, gadolinium chloride, gadolinium chloride hexahydrate and gadolinium nitrate hexahydrate.

7. The method of reducing dentinal sensitivity, comprising the steps of:
creating a formulation containing at least one of gadolinium and a gadolinium-containing compound; and
applying said formulation to a sensitive dentinal area.

8. The method as set forth in claim 7 wherein said formulation is one of a solution, a paste and a powder.

9. The method as set forth in claim 8 wherein said formulation contains gadolinium nitrate.

10. The method as set forth in claim 8 wherein the pH of said formulation is about 4.3.

11. The method as set forth in claim 8 wherein said formulation is a solution that contains at least one of water, sulfasalicyclic acid, sodium lauryl sulfate and alcohol.

12. The method as set forth in claim 11 wherein said solution contains sulfasalicyclic acid and water.

13. The method as set forth in claim 11 wherein said solution contains sulfasalicyclic acid and alcohol.

14. The method as set forth in claim 7 wherein the concentration of gadolinium or the gadolinium-containing compound is from about 1.5% to about 6%.
